(19) United States
(12) Patent Application Publication
(10) Pub. No.: US 2003/0190354 A1
(43) Pub. Date: Oct. 9, 2003

(54) Extended release composition comprising as active compound venlafaxine hydrochloride

(76) Inventor: Yoram Sela, Ra'anana (IL)

Correspondence Address: COUDERT BROTHERS, ATTN: LEWIS REFF, 1114 AVENUE OF THE AMERICAS, NEW YORK, NY, 10036, US

(21) Appl. No.: 10/406,777

(22) Filed: Apr. 3, 2003

(30) Foreign Application Priority Data
Apr. 9, 2002 (IL) 149.055

(51) Int. Cl. A61K 31/137; A61K 9/20; A61K 9/22

(52) U.S. Cl. 424/465; 514/649; 424/468

(57) ABSTRACT

A composition suitable for use in making extended release tablets of venlafaxine hydrochloride is described.

Venlafaxine Hydrochloride is an antidepressant having formula

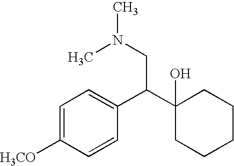

Being designated (R/S)-1-[2-(dimethylamino)-1-(4-methoxyphenyl) ethyl] cyclohexanol hydrochloride or (+)-1-[a] (dimethylamino) methyl) p-methoxybenzyl cyclohexanol hydrochloride having the empirical formula of C₁₇H₂₇NO₂ hydrochloride and molecular weight of 313.87

Venlafaxine hydrochloride is a white to off white crystalline solid with a solubility of 572 mg/ml in water (adjustment to ionic strength of 0.2 M with sodium chloride). Its octanol: water (0.2 M sodium chloride) partition coefficient 0.43. Effexor XR the brand product is formulated as an extended release capsule for once a day oral administration.

Controlled or extended release dosage forms of medicaments are conventionally produced as hydrogel matrix based tablets. At this technology the controlled release dosage forms are simply prepared by mixing the active material with the appropriate rate of controlling polymers and then that mixture is compressed into the desired controlled release tablets. The rate controlling polymers are normally termed as hydrogels. Examples of such polymers are cellulose ethers such as ethyl cellulose or hydroxypropylcellulose. Patents describing preparation methods of such dosage forms are described, for example, in U.S. Pat. No. 4,966,768 or 4,389,393.

In some cases, for example, with very water soluble active materials and with relatively high doses, it is not feasible to produce tablets which enable appropriate control on the drugs release. This is the case, for example with venlafaxine hydrochloride.

Several attempts were made, for example, to develop matrix based CR dosage forms of venlafaxine hcl. Attempts were performed by American Home Products Corporation, and as described in ER 0797991 A2 and U.S. Pat. No. 6,274,171 and as indicated in: “numerous attempts to produce extended release tablets by hydrogel technology proved to be fruitless because the compressed tablets were either physically unstable (poor compressibility or capping problems) or dissolved too rapidly in dissolution studies. Typically, the tablets prepared as hydrogel sustained release formulations gave 40-50% dissolution in 2 hrs, 60-70% dissolution at 4 hrs and 85-100% dissolution at 8 hrs.

In ALZA WO 94/27589 it is stated: “Venlafaxine need for ER but according to its high solubility, 570 mg/ml at 37° C., premature release of the drug from its dosage form might happen. As a result of convection motion of the imbibed fluid, and the hydrostatic pressure of the imbibed fluid coupled with the high solubility can result in the premature release of the drugs of the formula”.

In the above ALZA's application an osmotic pump is mentioned, designed to release Venlafaxine in a controlled manner.

In U.S. patent application Ser. No. 20010048943, an osmotic device designed to release venlafaxine hcl in a controlled manner, together with additional antipsychotic agent is mentioned. According to that application the venlafaxine is a least 80% released within 13 hrs.

Currently the marketed dosage form is microencapsulation based product. Said product is Effexor XR ER as described in WO 99/22724. The capsules contain 37.5, 75, or 150 mg of venlafaxine base. Said product is prepared in that a spheroid core is prepared by extruding and spheronizing a mixture of the drug with microcrystalline cellulose, and then coating it with an ethyl cellulose hydroxypropylmethylcellulose [HPMC] mixture.

This dosage form provides an extended release product with the following in vitro dissolution specifications:

| Time (hrs) | Average % Venlafaxine hcl released |
|---|---|
| 2 | <30 |
| 4 | 30-55 |
| 8 | 55-80 |
| 12 | 65-90 |
| 24 | >80 |

These dissolution characteristics are pH, RPM independent.

Despite the above mentioned, a controlled release dosage form of Venlafaxine HCl form was developed by Karma Pharm [Patent Application 146,462]. This form releases the extremely water soluble venlafaxine hydrochloride in a controlled manner up to 24 hrs and its dissolution specifications are comparable with the marketed microencapsulation based product. The release pattern of the venlafaxine tablets is PH, RPM independent.

In said previous invention the microencapsulation has been changed, i.e. it is being performed by layering the drug over an inert pareil core, and then coating it with an appropriate polymeric mixture.

Said invention consists in an extended release composition comprising as active compound Venlafaxine Hydrochloride, in which Venlafaxine Hydrochloride is coated on a non pareil inert core, which coated core is then coated with a polymeric layer which enables the controlled release of the Venlafaxine Hydrochloride.

However, said composition is not entirely satisfactory since microencapsulation is a relatively complicated and sensitive process regarding its development and manufacturability, thus a tablet dosage form was developed with a unique composition that controls the Venlafaxine HCl release at a rate similar to the microencapsulation product.

as it is too water soluble and thus a special combination was developed.

The present invention thus consists in an extended release composition comprising as active compound venlafaxine hydrochloride in a matrix tablet dosage form, in which the venlafaxine is mixed with a combination of hydrophilic and hydrophobic matrix forming components.

These matrix components are suitably combinations of hydrophilic polymers like hydroxypropyl methyl cellulose with high and low viscosity grades (100,000, 5 cp).

In addition, a combination of hydrophobic matrix forming components uses materials like ethyl cellulose (Ethocel 100 cp), together with glycerol behenate (compritol 888), their combination together with the methocels provide a unique combination which allows production of matrix based venlafaxine hcl controlled release tablets. Kollidon SR either in its dry form or as a suspension may be used as binder.

The venlafaxine hcl content is preferably 26-32% w/w of the dosage form. The composition according to the present invention comprises preferably the following hydrophilic component: hydroxypropyl methylcellulose [HPMC], having high and low viscosity grades.

The hydroxypropyl methylcellulose advantageously has a high viscosity of 100,000 cp and low viscosity HPMC with viscosity of 5 cp. Especially the ratio between the high and low viscosity components is between 2:1-6:1.

In said combination, the low viscosity grade HPMC tends to facilitate fast hydration of the high viscosity grade which act as a major release controlling component. The matrix forming hydrophilic components may be HPMC having a viscosity of 100,000 cp in a percentage of about 20% w/w.

The low viscosity methocel may, for example, be present in a percentage of 3-5% w/w of the composition.

A high viscous ethyl cellulose acts advantageously as hydrophobic matrix former component and as the hydrophobic matrix skeleton of the tablet and is preferably 7-9% w/w of the dosage form. The ration between HPMC and ethocel is advantageously between 2:1-5:1.

Compritol 888, the contents of which is preferably 12-16% w/w of the composition, may be used as a hydrophobic matrix former component with improved compressibility characteristics.

These tablets have dissolution profile independent at the tablet production technology, profile that complys with the innovators specifications for the microencapsulation product (Effexor XR), also have acceptable mechanical properties regarding hardness and friability.

Two granulation methods were used for the production of the tablets: the first was a regular one step granulation process, in which all excipients were blend together with the active, than wet granulated with Kollidon SR, dryed, milled and compressed into oral shape scored tablets.

The second granulation process was a two steps process, the first was wet granulation of the active material which was blended with the hydrophobic components: Ethocel, Compritol. Later on, the milled granulate was mixed with the hydrophilic components, the methocels and the lubricating components, syloid 244 and Mg stearate.

Unexpectedly, although these two processes are totally different and differences at the dissolution profiles were expected, practically the end results were the same and due to the unique hydrophilic/hydrophobic combination, the dissolution characteristics were found to be extremely robust and insensitive to the preparation process. The dissolution profile was insensitive to PH and RPM changes as well.

The present invention will now be illustrated with reference to the following examples without being limited by them:

### EXAMPLE NO. 1

| Components | |
|---|---|
| Venlafaxine hcl | 50 gr |
| Methocel K100M | 35 gr |
| Ethocel 100 cp | 15 gr |
| Avicel pH 101 | 20 gr |
| Kollicoat SR 30D | 33 gr |
| Compritol 888 | 20 gr |
| Syloid 244 | 1.5 gr |
| Mg stearate | 0.75 gr |

All composition components except of syloid and mg-stearate were mixed together and granulated with Kollicoat SR 30D suspension. The granules were dried in a fluid bed, 50° C. inlet air temperature for 45 minutes. The drug granules were screened through 0.63 mm screen, than blended with the syloid and mg-stearate and shaped into oval shape scored tablets with 75, 150 mg venlafaxine hcl.

### EXAMPLE NO. 2

| Components | |
|---|---|
| Venlafaxine hcl | 50 gr |
| Methocel K100M | 35 gr |
| Ethocel 100 cp | 15 gr |
| Avicel pH 101 | 20 gr |
| Kollicoat SFR 30D | 66 gr |
| Compritol 888 | 20 gr |
| Syloid 244 | 1.6 gr |
| Mg stearate | 0.8 gr |

The granulation, tableting process was as described in example no. 1.

### EXAMPLE NO. 3

| Components | |
|---|---|
| Stage 1 | |
| Venlafaxine hcl | 50 gr |
| Ethocel 100 cp | 15 gr |
| Avicel pH 200 | 10 gr |
| Compritol 888 | 25 gr |
| Kollidon SR | 10 gr |

Stage 1, wet granulation, than milling.

Stage 2, the milled granulates of stage 1 are dry blended with:

| | |
|---|---|
| Methocel K100M | 35 gr |
| Methocel 5 cp | 5 gr |
| Avicel pH 200 | 30 gr |
| Syloid 244 | 1.8 gr |
| Mg stearate | 0.9 gr |

After this second step the mixture was compressed into oval shaped scored tablets.

### EXAMPLE NO. 4

| Components | |
|---|---|
| Stage 1 | |
| Venlafaxine hcl | 50 gr |
| Ethocel 100 cp | 15 gr |
| Avicel pH 200 | 10 gr |
| Compritol 888 | 25 gr |
| Kollidon SR | 10 gr |
| Stage 2 | |
| Methocel K100M | 40 gr |
| Methocel 5 cp | 10 gr |
| Avicel pH 200 | 30 gr |
| Syloid 244 | 1.8 gr |
| Mg stearate | 0.9 gr |

The preparation method was two steps granulation and tableting process, as described in example no. 3.

The inactive materials full names are as follows: methocel K100M—Hydroxypropyl methylcellulose 208, 100 000 cp, Ethocel 100 cp—Ethyl cellulose 100 cp, Avicel—101/200—microcrystalline cellulose, Compritol 888—glycerol dibehenate EP, syloid 244—fumed amorphous silica, kollidon SR—polyvinylacetate—polyvinylpyrrolidon complex.

All of these examples have dissolution profile complies with the following specifications.

| Time hrs | average % Venlafaxine hcl release |
|---|---|
| 2 | <30 |
| 4 | 30-55 |
| 8 | 55-80 |
| 12 | 65-90 |
| 24 | >80 |

1. An extended release composition comprising as active compound venlafaxine hydrochloride in a matrix tablet dosage form, in which the venlafaxine is mixed with a combination of hydrophilic and hydrophobic matrix forming components.

2. Controlled release tablet dosage form having a unique combination of hydrophilic and hydrophobic matrix forming components which allows the controlled release of the extremely water soluble drug venlafaxine hcl, and also enables the production of tablets in an acceptable size and with acceptable physical characteristics.

3. Controlled release tablet according to claim 2 in which said tablets have dissolution characteristics similar to those of the microencapsulated commercial dosage form.

4. An extended release dosage form according to claim 1 wherein the venlafaxine hcl content is 26-32% w/w of the dosage form.

5. An extended release composition according to any of claims 1 to 4, comprising the following hydrophilic component: hydroxypropyl methylcellulose (HPMC), high and low viscosity grades.

6. Hydroxypropyl methylcellulose high viscosity grade according to claim 4 with viscosity of 100,000 cp and low viscosity HPMC with viscosity of 5 cp.

7. A combination of high and low viscosity grades of HPMC according to claim 5 in which the low viscosity grade HPMC tends to facilitate fast hydration of the high viscosity grade which act as a major release controlling component.

8. Methocels composition according to claim 6 or 7 in which the ratio between the high and low viscosity components is between 2:1-6:1.

9. Composition according to any of claims 1 to 8, in which the main matrix forming hydrophilic components is hydroxypropyl methylcellulose with high viscosity (100,000 cp).

10. Composition according to any of claims 6 to 8 in which the high viscosity grade HPMC presents in the formulation a percentage about 20% w/w.

11. Composition according to any of claims 6 to 10 in which the low viscosity methocel present in the formulation has a percentage of 3-5% w/w of the composition.

12. Composition according to any of claims 1 to 11 which contains highly viscous ethyl cellulose (100 cp) which acts as main hydrophobic matrix former component and acts as the hydrophobic matrix skeleton of the tablet.

13. Composition according to claim 12 in which the ethyl cellulose content is between 7-9% w/w of the dosage form.

14. Composition according to claim 2 in which the ratio between HPMC and ethocel is between 2:1-5:1.

15. Composition according to any of claims 1 to 14 which contains compritol 888 as a hydrophobic matrix former component with improved compressibility characteristics.

16. Composition according to claim 15 in which the compritol 888 content is 12-16% w/w of the composition.
